(19) United States
(12) Patent Application Publication
(10) Pub. No.: US 2010/0286488 A1
(43) Pub. Date: Nov. 11, 2010

(54) METHOD AND SYSTEM FOR USING A MOBILE DEVICE AS A PORTABLE PERSONAL TERMINAL FOR MEDICAL INFORMATION

(76) Inventors: Moshe Cohen, Netanya (IL); Rami Shmueli, Ramat Hasharon (IL)

Correspondence Address: SMITH FROHWEIN TEMPEL GREENLEE BLAHA, LLC, Two Ravinia Drive, Suite 700, ATLANTA, GA, 30346, US

(21) Appl. No.: 11/720,793

(22) PCT Filed: Aug. 25, 2005
(86) PCT No.: PCT/IL05/00921

§ 371 Date: Jun. 4, 2007

Related U.S. Application Data
(60) Provisional application No. 60/604,954, filed on Aug. 27, 2004.

(51) Int. Cl. A61B 5/00; G06Q 50/00

(52) U.S. Cl. 600/300; 705/2

(57) ABSTRACT

A medical information and support system that allows user's to gain access to their medical information on a mobile device terminal that interfaces to an application and server system through a wireless network. The mobile device may also interface to medical measurement systems to obtain vital and/or physiological measurements of the user. They system can trigger alarms if the physiological measurements are of concern. In addition, the mobile device can be used to provide instructions to a user to perform certain medical actions such as taking medication, as well as identifying the dosages and types of medication. The user can mobile device or through a web interface to the application and server system. In addition, health care entities may also access and/or modify the medical information through a web interface to the application and server system

### CROSS-REFERENCE TO RELATED APPLICATIONS

This application claims the benefit of the filing date and priority of U.S. Provisional Application for Patent which shares the same title as the present application, was filed with the United States Patent Office on Aug. 27, 2004 and has been assigned Ser. No. 60/604,954.

### BACKGROUND OF THE INVENTION

The present invention relates to the field of remote medical services and, more particularly, to using a mobile device, such as a cellular telephone, as a personal terminal and folder for storing and maintaining a patient's personal file containing medical or other personal information, processing the patient information, determining the patient's health condition and risk level, and activate a suitable application to reduce this risk.

We have all seen the television commercials of the medical alert device that is worn around a person's neck or wrist and that can be actuated to send out a medical alert or alarm. These devices are designed so that a patient can alert a healthcare provider, medical emergency personnel, or a personal care taker that the patient is in distress or is in need of medical attention. A problem that is inherent with this technology is that these devices do not contain any information pertaining to the medical history of the patient. What is needed in the art is a medical alert device that can provide alerts based on measurement data related to the status of a patient. For instance, if a patient is in distress or medical parameters are outside acceptable levels, it is desirable to have an automatic alert notification to be sent.

When a person is traveling or is simply out running errands, a condition can arise in which the individual may need medical attention. In many circumstances, the medical attention given to the patient could be tailored in a much more effective manner if the medical history or current medical information of the patient can be identified. Today, medical care providers must rely on the patient providing that information during an admission procedure or, having medical records transferred to the care providers. In many instances, this information simply cannot be obtained. For instance, if the patient is unconscious or unable to speak, the care provider will not be able to obtain the necessary information. In addition, the time-delay in obtaining pertinent information may render the information useless.

Another technology that is available in the market is reminders for a patient to take medication. This technology ranges from pillboxes that hold the medication for particular days of the week or days of the month and provides a physical indication to the patient that the medication has or has not yet been taken. Other more sophisticated technologies include timers that sound an alert when the patient is to take a medication. What is needed in the art is a device that not only reminds a patient to take their medication but, that can also instruct the patient as to what type of medication, the dosage to be taken, and other particular instructions about the medication (i.e., must be taken on an empty stomach etc) and that can also operate to verify that the patient has taken the medication. In addition, there is a need in the art for a device that can trigger a medical alert if the patient has missed taking medication or performing other medical actions, has incorrectly taken action, or has fulfilled an medical action.

Thus, there is a need in the art for a device that can provide medical information about a patient, operate to trigger alarms related to the present condition of the patient, provide instructions regarding medical actions that should be taken and providing alarms or alerts related to actions or inactions of the patient.

### BRIEF SUMMARY OF THE INVENTION

The present invention utilizes a personal device such as, but not limited to, a mobile device, a cellular telephone or a personal data assistant (PDA), a laptop, etc. as a personal terminal for maintaining a personal file of a patient's medical history or status. It should be noted that the terms “mobile device”, “personal device”, “cellular telephone”, “PDA” and laptop may be used interchangeably herein and the term mobile device may be used as a representative term for the above group. The personal file is a file that is particular to a patient and can contain information such as the medical, or other personal information, for managing the patient's medical routines, such as taking medicine, making the personal medical information available in case of emergency, measuring the patient's medical parameters through sensors integrated in the mobile device or by receiving the measurements from external measuring devices, for storing the patient's medical measurements and history, for synchronizing the personal information between the mobile device and an application and storage server through the mobile network, for continuously processing the patient's medical information received from the measuring devices, from the patient and from the application and storage server, determining the patient's health condition and risk level and activate the corresponding medical application in the mobile device, and for alerting the patient or others and activating the medical applications in the mobile device as a result of a change in one or more of the patient's medical parameters, or as a result of a change in the patient's medical file originated via the patient's physician or health care provider through the applications and storage servers, taking into account the patient's medical and personal information located in the mobile device or in the applications and storage servers. All these operations can be done regardless the location of the patient.

Embodiments of the present invention also provide location independent remote medical services (LIRMS). The LIRMS provides a user of the service with a combination of remote medical assistance that is independent of the current location of the user. This location independence aspect of the present invention is at least partially enabled due to the mobility feature of a cellular device. The cellular system operates to route a call to a cellular user regardless of the current location of the cellular user (even on a world-wide basis in some systems) as long as there is at least one cellular service provider in that area that has a service agreement with the home service provider of the user.

In one embodiment of the present invention, the mobile device contains a dedicated software program for managing all the personal file activities of the patient. This software is located as a part of the mobile device program memory or is downloaded to the mobile device SIM (or equivalent device) via the cellular network or at a service station.

Other objects, features, and advantages of the present invention will become apparent upon reading the following detailed description of the embodiments with the accompanying drawings and appended claims

### BRIEF DESCRIPTION OF THE SEVERAL VIEWS OF THE DRAWING

The invention is herein described, by way of example only, with reference to the accompanying drawings, wherein:

FIG. 1 is a block diagram of an exemplary communication system that is suitable for various embodiments of the present invention.

FIG. 2 is a block diagram of relevant modules of a mobile device according to an exemplary embodiment of the present invention.

FIG. 3 illustrates the flow chart of the data synchronization process between the mobile device and the application and storage server

FIG. 4 illustrates the flow chart of managing the patient medical routines

### DETAILED DESCRIPTION OF THE INVENTION

Turning now to the figures in which like numerals represent like elements throughout several views, exemplary embodiments of the present invention are described. For convenience, only some elements of the same group may be labeled with numerals. The purpose of the drawings is to describe exemplary embodiments and not for production. Therefore, features shown in the figures, although in and of themselves may be novel, are chosen for convenience and clarity of presentation only.

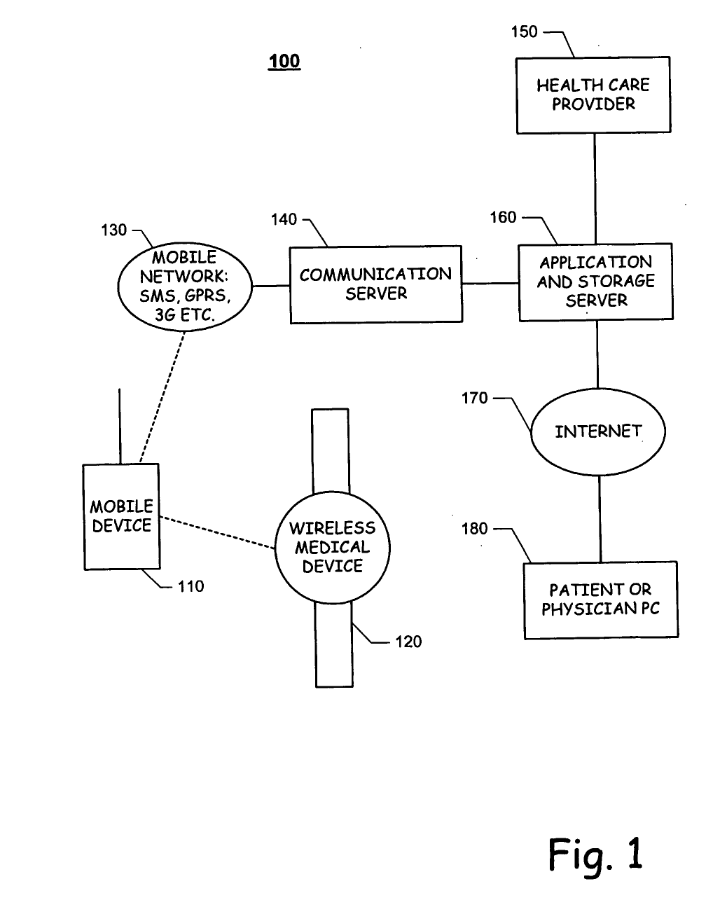

FIG. 1 is a block diagram of an exemplary communication system that is suitable for various embodiments of the present invention. The communication system 100 is shown as including a mobile cellular device 110, a wireless medical sensing device 120, a mobile or wireless network 130, a communication server 140, a health care provider (such as a call center or other facility) 150, an application and storage server (ASS) 160, a communication network 170 and a patient's or physician's PC or portable computer 180. The communication network 170 may include a variety of communication networks, including the Internet or any other wide area or global networks.

It should be noted that although FIG. 1 only illustrates one of each type of element, those skilled in the art will appreciate that multiple instances of each element or certain elements may be present. For example, one embodiment of the present invention may support several and/or a variety of mobile devices 110. Further, each mobile device 110 may be associated with one or more wireless medical devices 120. Likewise, the system may support multiple health care providers 150. Those skilled in the art will appreciate that many other variations may be implemented or that various aspects of the present invention can be incorporated into various embodiments of the present invention.

The mobile device 110 serves as a patient's access terminal to the system. The mobile device 110 can operate as a terminal for gaining access to the personal file of the patient, providing instructions or alerts to the patient, receiving action verifications from the patient and as an interface to a variety of medical devices. The mobile device 110 can be any of a variety of devices including cellular or mobile telephones, a PDAs that are equipped with cellular telephone capabilities, a computer or palm computer that includes cellular or mobile access, or any of a variety of wireless communication devices using various wireless communication technologies. The mobile device 110 is capable of communicating or interfacing with one or more wireless medical sensing devices 120 to support the various aspects of the present invention, and may also be equipped to perform the common functionality of the mobile device 110 (i.e., placing telephone calls in the case of a mobile telephone).

The mobile device 110 interfaces with one or more medical sensing devices and receives information or measurements. An exemplary wireless medical sensing device (WMSD) 120 may be a wrist-mount device for measuring physiological parameters of a user, such as temperature, heart rate, blood pressure, etc. An exemplary WMSD 120 is disclosed in PCT application number PCT/IL02/00995 having an international publication number of WO03/050643, the entire content of which is incorporated herein by reference. The WMSD 120 can be any of a variety of invasive and non-invasive devices, devise that come in direct contact with the patient or that make external measurements without contacting the client. Such devices include x-rays, MRI scans, blood analysis equipment, thermometers, etc. The WMSD 120 can communicate with the mobile device 110 via a standard wireless communication protocol such, as but not limited to, Bluetooth, IEEE 802.11 or other wireless or wired communication technologies. For instance, other embodiments of the present invention may use a propriety RF protocol, an infrared (IR) communication technology, a USB connector, etc. In some embodiments, the WMSD 120 can also receive transmissions from the mobile device 110 or other components within the system through the mobile device 110. Such transmissions can include data or instructions that can be utilized to control the operation of the WMSD 120.

The mobile network 130 may be based on a mobile network that is functional to carry data transportation or is otherwise capable of transferring data. Exemplary networks suitable for various embodiments of the present invention include GPRS, 3G, SMS and other similar technologies presently available or to be developed. The mobile network 130 provides an interface between the mobile devices 110 and the mobile communications server 140 and operates to provide data transmitted from the one or more mobile devices 110 to the mobile communication server 140 and ultimately to the application and storage server 160 and vise versa. The communication server 140 may reside at the cellular service provider for handling the data transportation over the cellular network 130 or can be provided by an independent third party. The communication server 140 may be connected directly to the ASS 160 or the communication server 140 may communicate with the ASS 160 via the Internet 170.

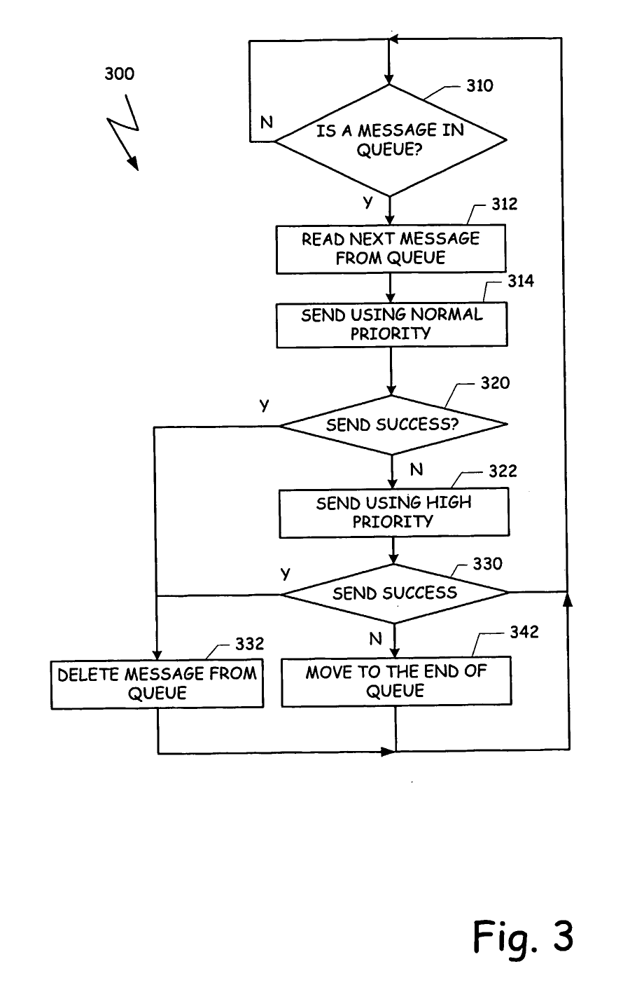

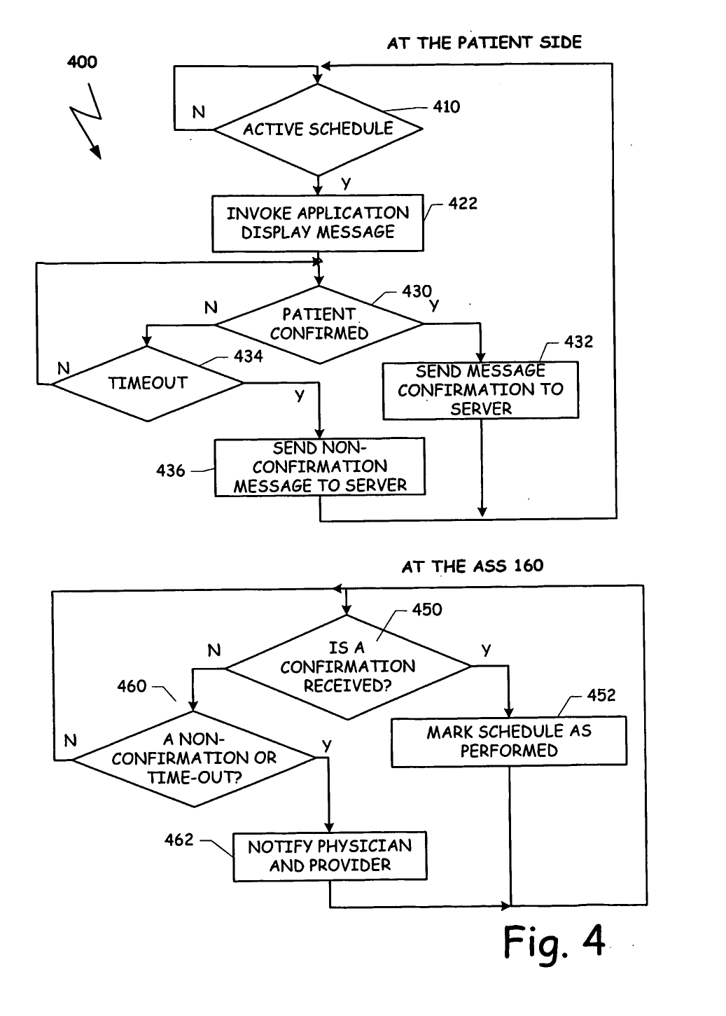

The ASS 160 is a medical server and houses personal medical folders for the various users or subscribers of the service. The ASS 160 also runs the applications that support the services of the present invention. The ASS 160 may communicate directly, or via the Internet 170, with one or more health care providers 150 including but not limited to medical call centers, MDs, etc. Furthermore, the ASS 160 may communicate via the Internet 170 with one or more patient's or physician's computers (PCs) 180 that are clients of the system 100. Further information regarding the ASS 160 is provided in conjunction with the discussion of FIGS. 3 and 4.

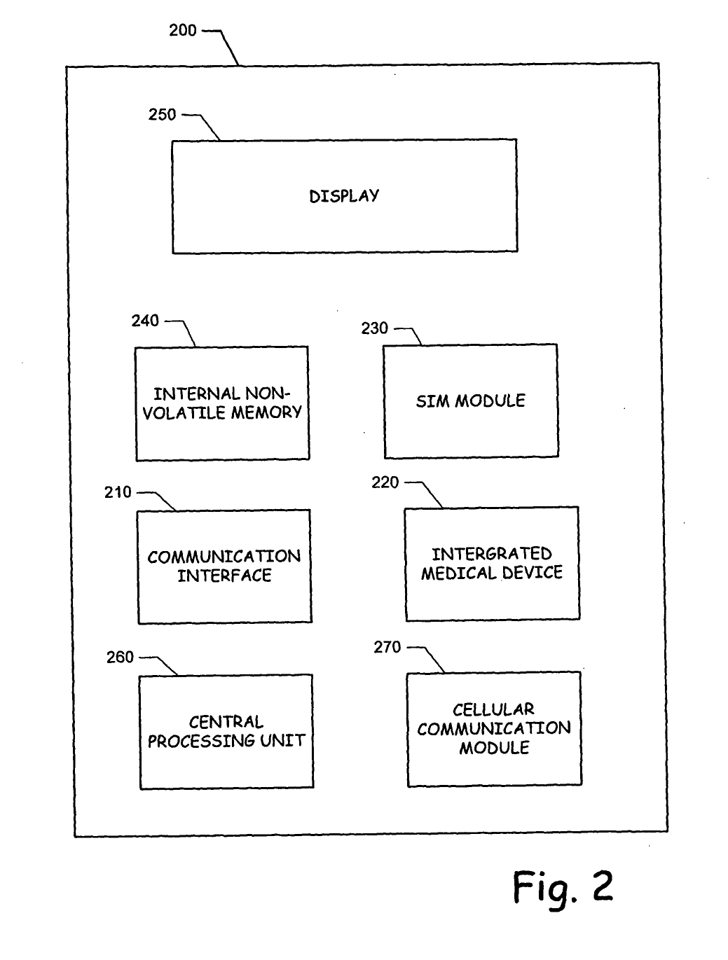

FIG. 2 is a block diagram of relevant modules or functional blocks of an exemplary mobile device according to an exemplary embodiment of the present invention. Among other modules, the mobile device 200 comprises: a communication interface (CI) 210, internal non-volatile memory (INVM) 240, SIM module 230 (or equivalent device), a display 250, a central processing unit 260 and a cellular communication module 270. In addition, mobile device 200 may comprise one or more integrated medical devices 220.

Communication module 210 is used to enable the mobile device 200 to communicate with one or more external devices. The communication module can be based on an RF module implementing the Bluetooth protocol; however, those skilled in the art will appreciate that other technologies may also be employed, such as but not limited to, an IR communication module, a wired communication module such as but not limited to USB, RS232, etc. The communication module 210 is used as the interface between a WMSD 120 (FIG. 1) and the mobile device 200.

The mobile device can include Integrated Medical Device 220 such as, but not limited to, one or more of the following: ECG, glucose meter, blood oxygen saturation meter, body temperature meter and blood pressure meter.

In one embodiment of the present invention, the INVM 240 and the SIM 230 may be the regular or standard modules that are included or sold in conjunction with the mobile device 200. However, in such an embodiment, the mobile device 200 should be reconfigured to dedicate or share a portion of the INVM 240 and/or the SIM 230 to store the software program that performs the functionality of aspects of the present invention, as well as to store the medical information and the personal folder that is associated with the user of the present invention.

Likewise, the display 250 can be the regular display that is included with the mobile device 200. However, when the mobile device 200 performs one of the medical applications, the display 250 may be used as the visual interface between the user and the medical application. It may display the results of the medical measurements, medical instructions, etc.

A Central Processing Unit 260 may be the regular CPU of the mobile device 200. This unit will execute the medical applications among the other mobile device applications.

The Cellular Communication Module 270 may be a regular cellular module of mobile device 200. This unit will be used for communicating between the medical applications and the ASS 160 (FIG. 1).

The following paragraphs teach some of the functionalities that are done by the present invention.

### Storing the Data Inside the Mobile Device

In an exemplary embodiment of the present invention, a portion of the mobile device's 200 internal non-volatile memory 240 is allocated for the medical or other data associated with a patient's personal file. This memory for the personal file can be a part of the internal non-volatile memory 240 of the mobile device 200 (FIG. 2), such as Flash memory, or a part of the SIM module non-volatile memory 230 of the mobile device (FIG. 2), or can be an additional plug-in non-volatile memory device (not shown in the drawings). In an exemplary embodiment, personal file is password protected. In a more particular embodiment, the personal file may be protected with multiple password levels so as to enable of access some of the data or the entire data according the password provided and the access rights associated with that password.

### Changing the Data and Synchronizing the Data Between the Mobile Device and the Application and Storage Servers

In one embodiment of the invention, a copy of the personal file for each patient or subscriber is maintained on both the patient's mobile device 110 and the ASS 160. The contents of both copies of the personal file are automatically synchronized between the mobile device 110 (FIG. 1) and the ASS 160 (FIG. 1). The medical data within the personal file can be modified by the patient using a personal file application module that is resident on the mobile device 110 (FIG. 1), or through other applications, such as a Web application that adds and changes the data in the ASS 160 (FIG. 1). The synchronization of the two copies ensures that the data in the mobile device 110 (FIG. 1) and in the ASS 160 (FIG. 1) is identical. The synchronization process contains a communication protocol to confirm the successful completion of every operation. Such synchronization techniques will be known to those skilled in the art and a variety of techniques may be utilized. In another embodiment of the invention, a master personal file may be stored on the ASS 160 and the mobile device 110 simply has continuous access to the personal file either through a down load request or through a browsing configuration. In this embodiment, any changes to the personal data that are initiated by using the mobile device 110 automatically changes the data in the personal file on the ASS 160.

An exemplary synchronization process 300 is described as a flow chart in FIG. 3.

The medical application at the mobile device 110 (FIG. 1) and/or the ASS 160 (FIG. 1) maintains a queue of messages that need to be sent to each other. Each message includes a time stamp and a priority level.

At step 310 the queue is checked and if the queue is empty, the process waits for a message to be entered to the queue. If a message is found in the queue or subsequently arrives into the queue, then the message originating side reads the message at step 312 and tries to establish a connection with the other end using the lowest cost method available 314, such as but not limited to 3G, UMTS, GPRS, MMS or SMS.

In case 320 that the connection is not established and the message priority is high, then the originating side will try to send the message via a different communication method 322 such as a dial up connection, for which the cellular operators provide higher service level guarantee.

In case 330 in which the communication is not established, the originating side can move the message to the last location in the queue 342 and returns to step 310.

In cases 320 or 330, if the message was sent successfully and acknowledged by the sender, the message will be removed from the originating side queue 332.

The receiving side application will use the time stamp of the received messages for resolving conflicts as a result of the messages not arriving immediately.

The communication between the applications will use a reliable protocol such as but not limited to TCP/IP.

### The Personal Medical Data

A copy of the patient's personal file is stored internally to, or otherwise associated with the mobile device 110. The personal file is used to provide the patient, or a health care provider, with relevant information about the patient. As such, the personal file can include a variety of information and those skilled in the art will certainly identify additional information beyond what is provided herein; however, some examples of the data may include the patient's personal information, such as medical history, medications, risk level, allergies and medical treatments, contact information related to the patient or emergency contacts for the patient, statistics and measurements related to the vital signs of the patient etc. The measurements data may contain the measurement parameter values and the raw data, such as ECG signals.

### Accessing the Personal Medical Data

In an exemplary embodiment, the mobile device may include a personal file application software module that enables the user or patient to access his or her personal file. The application software module may operate differently depending upon whether the user is accessing the personal file during an emergency situation or simply accessing the data for personal and/or daily use. If the user needs to access the personal file in response to an emergency, the personal file application software can provide access to a portion of the personal data using a dedicated common password, such as #*911. By inserting such a code, the personal emergency data will be displayed on the mobile device display. Using another password, the application software module will provide the patient access to all the personal file information. The data in the personal file can be displayed on the mobile device display, can be downloaded from the ASS 160 (FIG. 1) through the Web, can be printed from the mobile device through fax services supplied by the cellular operators when receiving a fax to a cellular telephone, or can be sent to a local printer through any communication interface, such as IR and Bluetooth. Those skilled in the art will appreciate that other mechanisms can be used for delivering or rendering the information to the user and the techniques presented herein, although they may be novel in and of themselves, are simply being provided as examples.

### Accessing and Updating the Personal File by Health Care Providers

The personal file, or portions of data within the personal file, can be accessed and updated by a third party, such as a health care provider 150 (FIG. 1), using Web applications connected to the ASS 160 or by sending the data in another format, such as XML, or any other format, to the ASS 160. The synchronization process assures that any modifications to the data in the ASS 160 will be updated in the mobile device 110 (FIG. 1). Thus, a Web application that is accessible to the healthcare provider can be invoked and provide a user interface enabling the healthcare provider to identify the patient, access the data, modify particular fields or portions of the data, and save the data into the personal file. Alternatively, the healthcare provider can send the modified or updated data via an email message, a formatted email message, a templated document, as raw data, through an FTP transfer, or many other techniques. The personal file can be protected through encryption and password access so that the healthcare provider is only able to access and change certain portions of the data.

Performing Measurements and Storing Results into the Personal File.

In addition to the medical information that is stored within a patient's personal file in the ASS 160 and the mobile device 110 and is accessible to the patient (FIG. 1), the patient can add additional information to the file. One example of such additional information is measurements that are obtained by using one or more medical devices 120 (FIG. 1). These measurements are transmitted from the medical device 120 to the mobile device 110 (FIG. 1) using a communication interface, such as RS232, IR, Bluetooth, ISM or other wireless or wired technologies. In addition, the medical devices may be equipped in such a mariner to transmit short messages, data messages or email messages to the mobile device that include such measurements. The measurements are then stored in the personal file on the mobile device 110 (FIG. 1) and then synchronized with the personal file in the ASS 160 (FIG. 1). The measurements can include a variety of information including the vital statistics or physiological status of the patient. For instance, the temperature, heart beat, blood pressure, skin resistance, or a variety of other physiological parameters can be measured and provided to the mobile device 110. Alternatively the patient can perform measurements using the integrated medical device 220 (FIG. 2).

### Managing the Patient'S Medical Routines

The personal file can also include data related to the management of the patient's medical treatment, such as the schedules, dosages, instructions and identification of medications. The management data can also include specific instructions to the patient to perform certain actions. For instance, the patient may periodically be requested to take his or her temperature, blood pressure and pulse. In addition, the patient may be requested to perform certain physical therapy activities, change bandages, etc. This management data can be entered into the personal file by the patient from the mobile device, can be entered by a patient, physician 180 or healthcare provider 150 (FIG. 1) through the ASS 160 (FIG. 1) or through other health care systems connected to the ASS 160. The personal file application software in the mobile device 110 (FIG. 1) in one embodiment, can be configured to verify that a patient has performed or completed a medical action that is included in the management data. This can be accomplished in a variety of ways, including but not limited to, prompting the user to press a button to confirm performance of a medical action or receiving an input from another device, such as a medical monitor, indicating an event has occurred. The medical action can be a one-time action or can be a periodical treatment. The confirmation obtained by the mobile device 110 can be sent to the ASS 160 (FIG. 1) and/or to the health care provider 150 (FIG. 1) connected to the ASS 160. This aspect of the present invention enables the health care providers 150 and physicians 180 to monitor the patient's performance of the requested actions.

An exemplary process 400 of managing the patient medical routines is described as a flow chart in FIG. 4. The process 400 may be divided in two sections or tasks. One task is implemented by the ASS 160 and the second one is implemented by mobile device 110.

The application at the mobile device 110 (FIG. 1) maintains a list of schedules. Each schedule contains a reminder message to be displayed to the user at a specified or required time. Optionally the schedule also includes an application, which needs to be executed at that time.

The ASS 160 (FIG. 1) maintains a list of medical routines for the patient. The routines can be run once and terminate routines or can be routines that run on a periodic or an a′ periodic basis.

When one of the schedules occur 410, an appropriate application is invoked. The invoked application can display 422 to the patient the required instructions and requests the patient to confirm the performance of the instructions or requests.

When the patient confirms the execution of the medical routine 430, a confirmation message is sent to the application and storage server 160 (FIG. 1) at step 432. Alternatively the medical application may also send a message indicating that the required action was not performed.

A timeout mechanism 434 and 436 may be added in order to indicate that the patient did not perform the requested activity on time.

In addition to the section that is disclosed above a parallel section of method 400 may run in the ASS 160. The ASS 160 waits for receiving a confirmation message 450. If a confirmation message was received 450, then the schedule activity is marked 452 at the application and storage server 160 (FIG. 1) as a performed action.

If the ASS 160 (FIG. 1) recognizes that an action was not performed as a result of receiving a non-confirmation message or as a result of not receiving a confirmation message within a defined timeout 460, it may generate a message to the healthcare provider 150 (FIG. 1) 462, to the physician or to the patient 180 (FIG. 1) via the Internet 170 (FIG. 1) or cellular network 140 (FIG. 1). An alternate embodiment of the present invention may use a timeout mechanism (not shown) also in the ASS 160 in order to reach a decision in step 460.

### Tracking the Patient'S Health Condition

The medical application will continuously process incoming data from one or more sources such as but not limited to medical measurements, patient's location, patient's medical file, patient's medical history, patient's medications, patient's laboratory test results, physician 180 (FIG. 1) and/or healthcare provider 150 (FIG. 1) observations and instructions and information received from the patient. This information is either located on the mobile device 110 (FIG. 1) or is imported from the application and storage server 160 (FIG. 1).

The medical application will continuously determine the patient's health condition and risk level and activate the appropriate medical application. This application will perform several tasks automatically in order to reduce the patient's risk level: notify one or more but not limited to the patient, the patient's relatives, the patient's physician 180 (FIG. 1), and healthcare provider 150 (FIG. 1) about the risk level, will present to the patient relevant general information about the risk and how to reduce the level of risk, and/or will give the patient specific recommendations and instructions how to reduce the level of risk.

For example, for a patient that has a history of breathing problems and lung disease, such as Asthma, in the medical file, the medical application will continuously track the patient location including altitude and will alert the patient in case of excessive altitude.

There will be several medical applications suitable for different situations, such as but not limited to normal activity, life style, elderly people, chronically ill, preventive, and sport or gym. For example, a Sport and Gym application interprets the measured heart rate in a different way than in normal activity and will be tolerant to higher heart rate results. The patient, the patient's physician 180 (FIG. 1) or the health-care provider 150 (FIG. 1) will have the ability to set up and change the current application in use.

The level of risk will be reduced by the health care provider 150 (FIG. 1) or the physician 180 (FIG. 1) or automatically by the application by processing the incoming data:

### Medical Alerts

Another aspect of the present invention is to provide medical alerts. The medical alerts can be triggered from a variety of actions or events and in general, are operative to inform or notify a patient, a health care provider for the patient, or a guardian or caretaker of the patient of a particular event or condition. As a non-limiting example, a medical alert can be triggered when a patient confirms or verifies that a medical action, such as taking a dosage of medication, has been performed. As another non-limiting example, a medical alert can be triggered when a patient measurement, such as heart rate, temperature, blood pressure, etc. meets or exceeds certain criteria. For instance, if a target or expected level is included in the patient's medical information, if the measurement varies from this value by a threshold amount an alarm may be triggered. As yet another non-limiting example, the medical alert can be triggered if the patient does not confirm that a requested medical action has been performed. The medical alerts may be provided to the patient, physicians 180 and/or the health care providers 150. One advantage of the present invention is that the medical alert may reach the user in any location in the world, in which the home service provider has an agreement. For instance, the personal file of a patient can be delivered to a cellular based mobile device by the ASS 160 simply dialing the mobile number or sending short messages or data packets to the mobile. Thus, regardless of the location of the user, the mobile device, once registered on the system, can be located and interfaced with the ASS 160.

The mobile device 110 (FIG. 1) contains a central processing unit 260 (FIG. 2) which is used for executing medical applications among the other mobile device applications. Every measurement taken by the patient, an input received locally from the patient or remotely from the application and storage servers 160 (FIG. 1), will be processed by the medical application, taking into account the patient medical history, medications information and relevant personal information. This information can be available locally on the mobile device 110 (FIG. 1) or can be imported from the application and storage servers 160 (FIG. 1). The medical application will respond and alert the patient, physician 180 (FIG. 1) and health care provider 150 (FIG. 1) according to the results. Additionally the results may change the mobile device active medical application or cause a new application to be loaded from the application and storage servers 160 (FIG. 1).

More information on the operation of the present invention can be found in the PCT application number PCT/IL02/00214 having the international publication number WO03/077,745 and in the PCT application number PCT/112004/000316 the entire contents of which are incorporated herein by reference. In the description and claims of the present application, each of the verbs, “comprise” “include” and “have”, and conjugates thereof, are used to indicate that the object or objects of the verb are not necessarily a complete listing of members, components, elements, or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art. The scope of the invention is limited only by the following claims.

What is claimed is:

1. A system for maintaining and providing medical data pertinent to a user, the system comprising:
a mobile device;
an application and storage server communicatively coupled to the mobile device and operative to maintain a personal file for the user, the personal file including the pertinent medical data; and
the mobile device being operative to access the personal file and render the data to the user.

2. The system of claim 1, wherein a copy of the personal file is maintained in memory of both the application and storage server and the mobile device; and the application and storage server and the mobile device are operative to synchronize the copies of the personal file.

3. The system of claim 1, wherein the mobile device is further operative to receive additional data to be included in the personal file.

4. The system of claim 2, wherein the application and storage server includes a third party interface, wherein a entity can access the personal file through the third party interface and modify the data within the personal file.

5. The system of claim 2, wherein the mobile device includes a software module that is operative to prompt the user to take certain medical actions based on the contents of the personal file.

6. The system of claim 5, wherein the software module is further operative to instruct a user to take a particular dosage of medication.

7. The system of claim 5, wherein the software module is further operative to receive an indicator that the user has performed the medical action.

8. The system of claim 7, wherein the software module is further operative to trigger an alarm if the software module has not received the indicator within a threshold period of time.

9. The system of claim 7, wherein the software module is further operative to trigger an alarm if the software module receives an indicator that the user has not performed the medical action.

10. The system of claim 2, wherein the mobile device includes a medical measurement device interface over which the mobile device can receive signals from a medical measurement device relevant to the user.

11. The system of claim 10, wherein the medical measurement device can measure one or more vital signs of the user and provide the measurement to the medical measurement device interface of the mobile device.

12. The system of claim 10, wherein the software module is further operative to determine whether to trigger an alarm based on the measurement and the synchronized personal file.

13. The system of claim 2 wherein the mobile device is communicatively coupled to the application and server system through a wireless telecommunications network that includes data transmission.

14. The system of claim 2, wherein the mobile device is communicatively coupled to the application and server through a digital cellular telecommunications network

15. The system of claim 10, wherein the medical measurement device is an integral part of the mobile device.

16. The system of claim 2, wherein a portion of the personal file is accessible by emergency personal.

17. The system of claim 1, wherein the mobile device is further operative to decide whether to invoke a medical application from the application and storage server.

18. The system of claim 17, wherein the decision which medical application to invoke is based on the measurements and the personal file.

19. The system of claim 17, wherein the decision which medical application to invoke is based on at lease one parameter selected from a group consisting of: location and user's input data and changes in the personal file.

20. The system of claim 1, wherein said mobile communication device is a cellular telephone.

21. The system of claim 1, wherein the application and storage server comprising a medical application that is adapted to determine the user health condition.

22. The system of claim 1, wherein the application and storage server comprising a medical application that is adapted to continually determine the user health condition.

23. The system of claim 1, wherein the application and storage server comprising a medical application that is adapted to continually determine the user risk level.

24. The system of claim 1, wherein the application and storage server comprises a medical application that is adapted to reducing the user risk level.

25. The system of claim 24, wherein reducing the user risk level is performed by informing medical services.

26. The system of claim 24, wherein reducing the user risk level is performed by informing user's relatives.

27. The system of claim 24, wherein reducing the user risk level is performed by giving the user specific recommendations and instructions.

28. The system of claim 1, wherein the application and storage server comprising a medical application that is adapted to be used in sport exercising period.

29. The system of claim 1, wherein the mobile device is at least one personal device selected from a group consisting of: a cellular telephone, a personal data assistant (PDA), a laptop and a personal computer.

30. The system of claim 1, wherein the decision which medical application to invoke from the application and storage server is done by the user.

31. The system of claim 1, wherein the decision which medical application to invoke from the application and storage server is done by a healthcare provider.

32. The system of claim 1, wherein the mobile device comprising a medical application that is adapted to determine the user health condition.

33. The system of claim 1, wherein the mobile device comprising a medical application that is adapted to continually determine the user health condition.

34. The system of claim 1, wherein the mobile device comprising a medical application that is adapted to continually determine the user risk level.

35. The system of claim 1, wherein the mobile device comprises a medical application that is adapted to reducing the user risk level.

36. The system of claim 1, wherein the mobile device comprising a medical application that is adapted to be used in sport exercising period.
